(19) United States
(12) Patent Application Publication
(10) Pub. No.: US 2007/0074301 A1
(43) Pub. Date: Mar. 29, 2007

(54) Novel fluorescent Cichlid and method for producing thereof

(76) Inventor: Huai Jen Tsai, Taipei (TW)

Correspondence Address: HARNESS, DICKEY & PIERCE, P.L.C., P.O. BOX 8910, RESTON, VA, 20195, US

(21) Appl. No.: 11/235,539

(22) Filed: Sep. 27, 2005

(51) Int. Cl. A01K 67/027

(52) U.S. Cl. 800/20

(57) ABSTRACT

The invention relates to a method for producing fluorescent Cichlid. The invention also relates to novel fluorescent Cichlid.

### FIELD OF THE INVENTION

This invention relates to a novel fluorescent Cichlid and a method for producing the fluorescent Cichlid.

### BACKGROUND OF THE INVENTION

Ornamental fish is part of the fishery business and has a global market. Therefore, using recombinant DNA and transgenic techniques to modify the phenotypes of ornamental fish has great market value.

Transgenic fish studies make use of genes that are driven by both heterologous and homologous sources of regulatory element, and originate from constitutive or tissue-specific expression genes. Control elements include genes from antifreeze protein, mouse metallothionein, chicken δ-crystalline, carp β-actin, salmon histone H3 and carp α-globin and so on. However, there are important drawbacks to the use of these DNA elements in transgenic fish, including low expression efficiency and the mosaic expression of transgene patterns.

It is successful to express a transgene in F0 generation and the continuous two generations by using a DNA construct which contains the ITR sequences in both termini to increase the transgenic efficiency (Chi-Yuan Chou et al., 2001, Transgenic Research 10: 303-315). Although the green fluorescent transgenic fish has been made, the methods and conditions for producing other fluorescent or other species are different, including different gene construct strategies, gene expression stability, genetic inheritance and other uncertain factors. Therefore, the skill can not be easily derived from the person skilled in the art.

With the developments of transgenic techniques, efforts have been made to produce various transgenic Cichlid especially on the growth enhancement of Cichlidae and biodiversity research thereof; however, a problem with application of this methodology to cichlids is that transgenic techniques have been hardly established for these fish so far.

Ken et al reported that when the mMT-hGH gene was micro-injected into Cichlid fish (Cichlasoma nigrofasciutum) eggs at 1-32 cell stages, the gene sequences can be integrated into the genome of the embryos. (Ken C F, Liao C F, Hsu Y L and Wu J L: International Symposium on RBA, April 22-27, Tapie, China, Program and Abstract, p 53, 1991).

Cichlid (Cichlidae), a family of Acanthopterygian fishes, is related to the perches and wrasses, and confined to the fresh and brackish waters of Central and South America, Africa, Syria, India and Ceylon. It has recently assumed special importance through the large number of genera and species, many of them showing extraordinary modifications of the dentition, which have been discovered in tropical Africa, especially in the great lakes Victoria, Tanganyika and Nyasa. About 180 species are known from Africa (with Syria and Madagascar), 150 species from America, India and Ceylon.

Cichlids come in a surprising diversity of shapes, sizes and colors. They share a few common characteristics, including life in freshwater and advanced forms of parental care of their progeny.

Habitat Distribution—Cichlids are found in almost every possible body of freshwater within their geographic range including rivers, lakes, swamps, and even ditches and puddles. They are not found at high elevations and generally require water warmer than about 20° C. (68° F.).

Diversity of Size and Form—There are large predatory cichlids like the peacock bass (Cichla ocellarus) of South America and tiny secretive cichlids (often called “dwarfs”) found in many places, including the kribensis group from West Africa (e.g., Pelvicachromis pulcher) and the genus Apistogramma from South America. Further, there are herbivores (plant-eaters) like the mbuna of Lake Malawi and carnivores like the slender pike-cichlids of the Amazon.

The popularity of cichlids among aquarists likely stems from three things: many are easy to keep, there are so many kinds and they do interesting things. With few exceptions cichlids can be kept and bred in aquaria. Hundreds of species are currently available in the hobby and many can be kept with a minimum of equipment in aquaria ranging from 10 gallons on up. Some require specialized care and are not for the beginner, but many others are easy to keep and breed. All cichlids provide some form of parental care, meaning that one or both parents tend to the eggs and/or young after laying. Cichlids are devoted parents and watching a pair of cichlids doing their thing is hard to beat.

In general, the body color of tropical fish is more fascinating than that of natural Cichlids. In addition, it is really hard to make natural Cichlids with charming color like tropical fish. Further, for the customers, the breed of freshwater fish is easier than that of tropical fish. There is a need to produce a Cichlid with fascinating color (such as fluorescence) to broad Cichlid market and meet customer desire.

### SUMMARY OF THE INVENTION

This invention provides a method for producing fluorescent Cichlid comprising: (a) constructing a plasmid containing inverted terminal repeats (ITR), α-actin or β-actin promoter, a fluorescent gene, SV40 poly A and ITR from upstream to downstream; (b) microinjecting the plasmid construct into fertilized eggs of Cichlid; and (c) selecting and hatching the fluorescent Cichlid.

This invention also provides a fluorescent Cichlid which is produced from the method of the invention.

### BRIEF DESCRIPTION OF THE DRAWINGS

FIG. 1 illustrates the map of plasmid pDsRed2-1-ITR. The restriction site for plasmid linearization is Not1.

FIG. 2 illustrates the embryos of transgenic Angelfish microinjecting 48 hours after microinjection.

FIG. 3 illustrates the transgenic Angelfish expressing significant red fluorescence 48 hours after hatching. The left figure shows the Angelfish under common light and the right figure shows the Angelfish under fluorescent light.

### DETAILED DESCRIPTION OF THE INVENTION

The present invention will be described more fully hereinafter with reference to the attached drawings, in which some, but not all embodiments of the invention are shown. Indeed, these inventions may be embodied in different forms and should not be construed as limited to the embodiments set forth herein.

By “promoter” is intended a DNA sequence that directs the transcription of a gene. Usually, it is a regulatory region of DNA comprising a TATA box capable of directing RNA polymerase II to initiate RNA synthesis at the appropriate transcription initiation site for a particular coding sequence. Typically a promoter is located in the 5 prime region of a gene, proximal to the transcriptional start site of a coding sequence. A promoter may additionally comprise other promoter and enhancer recognition sequences generally positioned upstream or 5 prime to the TATA box, referred to as upstream promoter and enhancer elements, which influence the transcription initiation rate.

By “enhancer” is a DNA regulatory element that can increase efficiency of transcription regardless of the distance or orientation of the enhancer relative to the start site of transcription.

The present invention provides a method for producing fluorescent Cichlid comprising: (a) constructing a plasmid containing inverted terminal repeats (ITR), α-actin or β-actin promoter, a fluorescent gene, SV40 poly A and ITR from upstream to downstream; (b) microinjecting the plasmid construct into fertilized eggs of Cichlid; and (c) hatching the fertilized eggs to the fluorescent Cichlid. The preferred plasmid is

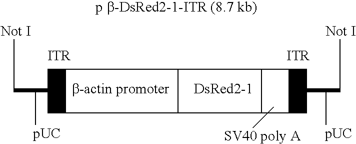

The fluorescent gene is not limited to red fluorescent gene but orange fluorescent gene, yellow fluorescent gene, green fluorescent gene, blue fluorescent gene, cyan fluorescent gene or other fluorescent genes. In a preferred embodiment, the fluorescent gene is red fluorescent gene from pDsRed2-1 and the promoter is β-actin promoter.

The red fluorescent gene can be purchased from BD Bioscience Clontech. In the embodiment of the invention, pDsRed2-1 is used as the source of the red fluorescent gene. pDsRed2-1 encodes DsRed2, a DsRed variant engineered for faster maturation and lower non-specific aggregation. DsRed2 contains a series of silent base-pair changes that correspond to human codon-usage preferences for high expression in mammalian cells. In cell cultures when DsRed2 is expressed constitutively, red-emitting cells can be detected by fluorescence microscopy within 24 hours of transfection. Large insoluble aggregates of protein, often observed in bacterial and mammalian cell systems expressing DsRed1, are dramatically reduced in cells expressing DsRed2. The faster-maturing, more soluble red fluorescent protein is also well tolerated by host cells. That is, mammalian cell cultures transfected with DsRed2 show no obvious signs of reduced viability-in those cell lines tested, cells expressing DsRed2 display the same morphology (e.g., adherence, light-refraction) and growth characteristics as non-transfected controls. pDsRed2-1 is a promoterless DsRed2 vector that can be used to monitor transcription from different promoters and promoter/enhancer combinations inserted into the multiple cloning site (MCS).

In the method of producing fluorescent Cichlid of the invention, the plasmid is linearized by the restriction enzyme NotI. Injecting of appropriate amount of NotI-linearized plasmid construct into the fertilized eggs is sufficient to introduce transgene into germ cell of Cichlid. The concentration of the plasmid injected into the eggs is 30 ng/ml. The amount of the injected plasmid is from about 10 pl to about 50 pl, preferably from 10 pl to 30 pl, and more preferably from 15 pl to 25 pl.

In this invention, the Cichlid is selected from the members of family Cichlidae. In a preferred embodiment, the Cichlid is selected from the group consisting of Angelfish, Discus and South American Cichlid. In a more preferred embodiment, the Cichlid is selected from the group consisting of Pterophyllum scalare, Symphysodon aequifasciatus spp., Acarichtbys beckeli, Aeguidens rivulatus, Cichlasoma carpinte, Astronauts ocellatus, Cichla ocellaris, Cichla temensis, Cichasoma citrinellum, Cichasoma cyanoguttatum, Cichasoma synspilus, Cichasoma meeki, Cichasoma nigrofasciatum, Cichasoma severum, Cichasoma temporale, Cichasoma tetracanthus, Geophagus balzanii, Geophagus steindachneri, Geophagus surinamensis, Hemichromis lifalili, Lepomis gibbosus, Lepomis megalotis and Uaru amphiacanthoides. In a further more preferred embodiment, the Cichlid is Cichasoma citrinellum and Cichasoma synspilus. In the most preferred embodiment, the Cichlid is Pterophyllum scalare, also called Angelfish.

The method of the present invention further comprises the steps of (a) crossing the fluorescent Cichlid with wild type of Cichlid, or (b) crossing the fluorescent Cichlid with different or same species of the fluorescent Cichlid to produce a fluorescent offspring Cichlid. In a preferred embodiment, the fluorescent Cichlid is fluorescent Cichasoma citrinellum, the different species of the fluorescent Cichlid is fluorescent Cichasoma synspilus and the fluorescent offspring Cichlid is fluorescent blood parrot (Cichasoma citrinellum).

The method of the invention provides five improvements over other methods currently available:
1. The main body of the nucleic acid fragment of the invention is a commercially available construct.
2. The nucleic acid fragment of the invention enables the Cichlid to emit fluorescence throughout its systemic skeletal muscle.
3. The method of the invention, which comprises microinjecting the transgene construct into fertilized eggs, enables the transgenic Cichlid to emit fluorescence throughout whole body including skeletal muscle at a higher ratio with better quality.
4. The heterologous transgenic fish stably passes the transgene to the next generation. Thus natural breeding could be used to maintain the transgenic line and reduce the breeding cost.
5. The fluorescence of the transgenic Cichlid, which is emitted throughout whole body including skeletal muscle, can be easily seen by naked eyes. The red fluorescence is further intensified under light source of shorter wavelength, providing a higher entertainment value to ornamental fish.

The present invention also provides a fluorescent Cichlid which is produced from the method of the invention. The fluorescent Cichlid can express systemic fluorescence. The Cichlid is selected from the members of family Cichlidae. In a preferred embodiment, the Cichlid is selected form the group consisting of Angelfish, Discus and South American Cichlid. In a more preferred embodiment, the Cichlid is selected form the group consisting of Pterophyllum scalare, Symphysodon aequifasciatus spp., Acarichtbys beckeli, Aeguidens rivulatus, Cichlasoma carpinte, Astronauts ocellatus, Cichla ocellaris, Cichla temensis, Cichasoma citrinellum, Cichasoma cyanoguttatum, Cichasoma synspilus, Cichasoma meeki, Cichasoma nigrofasciatum, Cichasoma severum, Cichasoma temporale, Cichasoma tetracanthus, Geophagus balzanii, Geophagus steindachneri, Geophagus surinamensis, Hemichromis lifalili, Lepomis gibbosus, Lepomis megalotis and Uaru amphiacanthoides. In a further more preferred embodiment, the Cichlid is Cichasoma citrinellum, Cichasoma synspilus or blood parrot (Cichasoma citrinellum) such as untailed blood parrot, red diamond parrot, pearl parrot, unicorn blood parrot, blood parrot “Red and White”, blood parrot “rose empress”, blood parrot “king-kong,” etc. In the most preferred embodiment, the Cichlid is Pterophyllum scalare, also called Angelfish.

Further, the fluorescent Cichlid can express the fluorescent gene such as red fluorescent gene, orange fluorescent gene, yellow fluorescent gene, green fluorescent gene, blue fluorescent gene, cyan fluorescent gene or other fluorescent gene. The preferred fluorescent Cichlid has systemic red fluorescence from pDsRed2-1.

### EXAMPLES

The examples below are non-limiting and are merely representative of various aspects and features of the present invention.

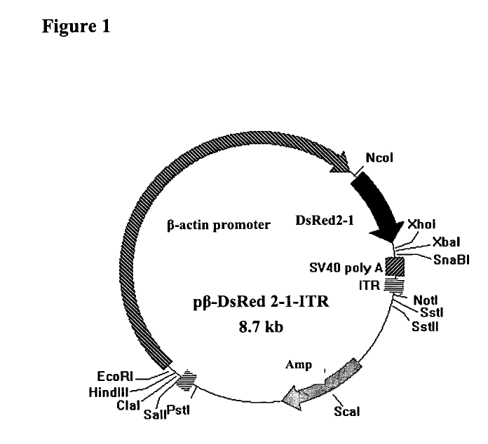

The method for producing Cichlid with red fluorescence:
1. Commercially available plasmid construct, pDsRed2-1 (Clontech) was used to generate the expression vector.
2. The DsRed fragment was derived from plasmid pDsRED2-1. The CMV promoter and two adeno-associated virus inverted terminal repeats (ITR) were ligated to the DsRed fragment to produce plasmid construct pDsRed2-1-ITR. The plasmid construct pDsRed2-1-ITR has shown higher expression stability.
3. The CMV promoter was replaced by β-actin promoter, thus the novel plasmid construct pβ-DsRed2-1-ITR as shown in FIG. 1 was generated.

The β-actin promoter was obtained by digesting plasmid construct pOBA-109 with restriction enzymes NcoI and EcoRI. NcoI was used first, ends were filled in, and a subsequent digestion with EcoRI provided a 4 kb fragment.

The CMV promoter was cut out by digesting the construct pDsRed2-1-ITR with restriction enzymes BamHI and SalI. Digestion with BamHI and SalI provided a 4.7 kb fragment. Then, the β-actin promoter of Cichlid was inserted into the plasmid construct, pDsRed2-1-ITR, at the position where the CMV promoter was cut out. The resulting plasmid construct had two 145 bp adeno-associated virus inverted terminal repeats (ITR). One ITR was located at the 3′ end of SV40 poly A. The other was located at the 5′ end of the β-actin promoter.

As illustrated in FIG. 1, the resulted plasmid construct, pβ-DsRed2-1-ITR, had a total length of 8.7 kb. pβ-DsRed2-1-ITR contained (1) the Cichlid β-actin gene promoter (for ubiquitous expression of whole body); (2) red fluorescent gene; (3) adeno-associated virus inverted terminal repeats; and (4) pUC plasmid construct basis.

The plasmid construct pβ-DsRed2-1-ITR was transformed into Escherichia coli 5α.
4. Linearization of the plasmid construct by using restriction enzyme:

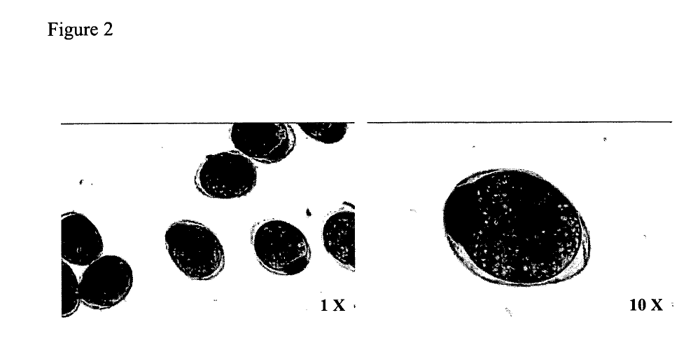

As illustrated in FIG. 1, appropriate amount of pβ-DsRed2-1-ITR was digested with proportional amount of Not I restriction enzyme. A small fraction of the digested product was analyzed by agarose gel electrophoresis to verify its linearity. The fragment length was 8.7 kb as expected. Then, the digested DNA products were extracted by a solution containing phenol:chloroform (1:1), precipitated by ethanol, air dried, then dissolved in PBS at a concentration of 30 ng/ml, which would be used for later cytoplasmic microinjection.
5. Cytoplasmic microinjection
   a. Collecting fertilized eggs: Set one male and one female mature Angelfish in an aquarium at 28° C. for natural mating. The semicircle-shaped plastic pipe (PVC, diameter 6 cm) was placed under the bottom. The Angelfish laid eggs on the plastic pipe. After laying eggs for 15-20 min, the eggs were sucked by sucker directly and collected in distilled water at 10° C.
   b. Microinjection: The linearized construct was quantified and dissolved in 5×PBS with phenol red at the desired concentration. DNA was picked up by micro-capillary of microinjector (Drummond) wherein the scope of injection needle opening width was 2-10 μm. As micro-needle enters the cell cytoplasm, the amount of DNA injected was around 20-30 pl.
   c. Hatching fertilized eggs: Injected eggs were rinsed with sterilized solution, cultured in incubator wherein the temperature was 26° C. The fluorescence could be observed in the developing embryos after 48 hours as illustrated in FIG. 2.
6. Fluorescent microscopy observation:

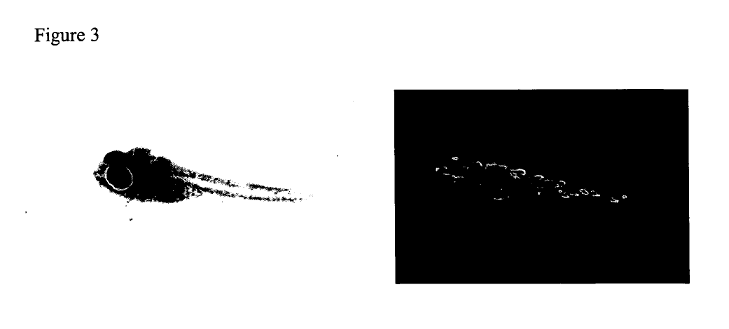

The injected embryos were placed in water. The distribution and intensity of the red fluorescence is observed under fluorescence microscope (Leica MZ-12; Fluorescence System light source Hg 100 W; main emission wavelength 558 nm, and main absorption wavelength 583 nm, filter set RFP-Plus; photography system MPS60). The fluorescent Angelfish was shown in FIG. 3.
7. Germ-line transmission of transgene:

To get the progeny that exhibited uniform fluorescence, the red fluorescent Cichlid originated from embryos microinjected with pβ-DsRed2-1-ITR fragment were mated with wild type, The F1 with fluorescence expression was again mated with wild type to obtain the F2 progeny, which all exhibited red fluorescent expression, and can be readily distinguished from fish without fluorescence expression. The difference between transgenic fluorescent Cichlid and wild type Cichlid could be better determined under blue light.

The DNA fragment of the invention could be modified to carry other fluorescent genes, and thus fish with different fluorescence could be produced.

Other transgene constructs comprising other fluorescence genes may be introduced to Cichlid eggs along with red fluorescence to make fish with various body colors.

The Cichlid of the invention can be broadly used in researches in medicine and other fields of life science, for example, cell fusions, cloning, nuclear transfer, cell motility, cell targeting, and embryonic development.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method for producing fluorescent Cichlid comprising:
(a) constructing a plasmid containing inverted terminal repeats (ITR), α-actin or β-actin promoter, a fluorescent gene, SV40 poly A and ITR from upstream to downstream;
(b) microinjecting the plasmid construct into fertilized eggs of Cichlid; and
(c) selecting and hatching the fluorescent Cichlid.

2. The method of claim 1, wherein the plasmid is pβ-DsRed2-1-ITR as illustrated in FIG. 1.

3. The method of claim 1, wherein the fluorescent gene is selected from the group consisting of red fluorescent gene, orange fluorescent gene, yellow fluorescent gene, green fluorescent gene, blue fluorescent gene and cyan fluorescent gene.

4. The method of claim 3, wherein the red fluorescent gene is from pDsRed2-1.

5. The method of claim 1, wherein the Cichlid is selected from family Cichlidae.

6. The method of claim 5, wherein the Cichlid is selected from the group consisting of Angelfish, Discus and South American Cichlid.

7. The method of claim 6, wherein the Cichlid is selected from the group consisting of Pterophyllum scalare, Symphysodon aequifasciatus spp., Acarichtbys beckeli, Aeguidens rivulatus, Cichlasoma carpinte, Astronauts ocellatus, Cichla ocellaris, Cichla temensis, Cichasoma citrinellum, Cichasoma cyanoguttatum, Cichasoma synspilus, Cichasoma meeki, Cichasoma nigrofasciatum, Cichasoma severum, Cichasoma temporale, Cichasoma tetracanthus, Geophagus balzanii, Geophagus steindachneri, Geophagus surinamensis, Hemichromis lifalili, Lepomis gibbosus, Lepomis megalotis and Uaru amphiacanthoides.

8. The method of claim 2, wherein the plasmid is linearized by using restriction enzyme Not I.

9. The method of claim 1, wherein the plasmid used to inject into the egg is in the amount of 10-50 pl.

10. The method of claim 1, which further comprises the step of (a) crossing the fluorescent Cichlid with wild type of Cichlid, or (b) crossing the fluorescent Cichlid with different or same species of the fluorescent Cichlid to produce an offspring fluorescent Cichlid.

11. A fluorescent Cichlid which is produced from the method of claim 1.

12. The Cichlid of claim 11, which is selected from Family Cichlidae.

13. The Cichlid of claim 11, which is selected from the group consisting of Angelfish, Discus and South American Cichlid.

14. The Cichlid of claim 11, which is selected from the group consisting of Pterophyllum scalare, Symphysodon aequifasciatus spp., Acarichtbys beckeli, Aeguidens rivulatus, Cichlasoma carpinte, Astronauts ocellatus, Cichla ocellaris, Cichla temensis, Cichasoma citrinellum, Cichasoma cyanoguttatum, Cichasoma synspilus, Cichasoma meeki, Cichasoma nigrofasciatum, Cichasoma severum, Cichasoma temporale, Cichasoma tetracanthus, Geophagus balzanii, Geophagus steindachneri, Geophagus surinamensis, Hemichromis lifalili, Lepomis gibbosus, Lepomis megalotis and Uaru amphiacanthoides.

15. The Cichlid of claim 14, which is Pterophyllum scalare.

16. The fluorescent Cichlid of claim 11, which expresses the fluorescent gene selected from the group consisting of red fluorescent gene, orange fluorescent gene, yellow fluorescent gene, green fluorescent gene, blue fluorescent gene, cyan fluorescent gene and other fluorescent gene.

17. A fluorescent Cichlid which is produced from the method of claim 10.

18. The Cichlid of claim 17, which is selected from the group consisting of Cichasoma citrinellum, Cichasoma cyanoguttatum, Cichasoma synspilus, Cichasoma meeki, Cichasoma nigrofasciatum, Cichasoma severum, Cichasoma temporal, and Cichasoma tetracanthus.

19. The Cichlid of claim 17, wherein the fluorescent Cichlid is fluorescent Cichasoma citrinellum and the different species of the fluorescent Cichlid is fluorescent Cichasoma synspilus.

20. The Cichlid of claim 19, wherein the offspring fluorescent Cichlid is fluorescent blood parrot (Cichasoma citrinellum).
